(19) United States
(12) Patent Application Publication
(10) Pub. No.: US 2023/0042040 A1
(43) Pub. Date: Feb. 9, 2023

(54) METHODS AND APPARATUS FOR QUANTUM CHEMISTRY CALCULATIONS ON A QUANTUM COMPUTER

(71) Applicant: Xanadu Quantum Technologies Inc., Toronto (CA)

(72) Inventors: Juan Miguel ARRAZOLA, Toronto (CA); Olivia Di MATTEO, Hamilton (CA)

(73) Assignee: Xanadu Quantum Technologies Inc., Toronto (CA)

(21) Appl. No.: 17/845,344

(22) Filed: Jun. 21, 2022

Related U.S. Application Data
(60) Provisional application No. 63/214,495, filed on Jun. 24, 2021.

(51) Int. Cl. G16C 10/00; G06N 10/60; G06N 10/20

(52) U.S. Cl. CPC G16C 10/00; G06N 10/60; G06N 10/20

(57) ABSTRACT

A quantum chemistry method includes causing display, via a processor, of a representation of a plurality of controlled single-excitation quantum gates. A selection of a subset of controlled single-excitation quantum gates from the plurality of controlled single-excitation quantum gates is received at the processor. A particle-preserving unitary for a quantum chemistry simulation is identified based on the selected subset of controlled single-excitation quantum gates. At least one controlled single-excitation quantum gate from the plurality of controlled single-excitation quantum gates can be configured to apply a Givens rotation.

### CROSS-REFERENCE TO RELATED PATENT APPLICATIONS

This application claims the benefit of, and priority to, U.S. Provisional Application No. 63/214,495, filed Jun. 24, 2021 and titled “Methods and Apparatus for Quantum Chemistry Calculations on a Quantum Computer,” the entire content of which is incorporated herein by reference for all purposes.

### FIELD

The present disclosure is related to quantum computing, and more specifically, to the simulation of quantum circuits for quantum chemistry applications.

### BACKGROUND

Quantum chemistry is a branch of chemistry that involves the application of quantum mechanics to chemical systems. Major goals of quantum chemistry include increasing the accuracy of the results for small molecular systems, and increasing the size of large molecules that can be processed, which is limited by scaling considerations in that the computation time increases as a power of the number of atoms.

### SUMMARY

In some embodiments, a method includes causing display, via a processor, of a representation of each controlled single-excitation quantum gate from a plurality of controlled single-excitation quantum gates. A selection of a subset of controlled single-excitation quantum gates from the plurality of controlled single-excitation quantum gates is received at the processor. A particle-preserving unitary for a quantum chemistry simulation is identified based on the selected subset of controlled single-excitation quantum gates. At least one controlled single-excitation quantum gate from the plurality of controlled single-excitation quantum gates can be configured to apply a Givens rotation.

### BRIEF DESCRIPTION OF THE DRAWINGS

The drawings primarily are for illustration purposes and are not intended to limit the scope of the subject matter described herein. The drawings are not necessarily to scale; in some instances, various aspects of the disclosed subject matter disclosed herein may be shown exaggerated or enlarged in the drawings to facilitate an understanding of different features. In the drawings, like reference characters generally refer to like features (e.g., functionally similar and/or structurally similar elements).

FIG. 1 shows an example of a Jordan-Wigner representation of states with a fixed number of particles.

FIG. 2 shows example three-qubit controlled single-excitation gates that are universal for particle-conserving unitaries, and a double-excitation gate, according to some embodiments.

FIG. 3 is a diagram showing that any pair of states for a fixed number of particles can be related by an excitation, according to some embodiments.

FIG. 4 is a diagram of a single-excitation gate G that can act non-trivially on many states, according to some embodiments.

FIG. 5 shows a circuit implementing a triple-excitation rotation that can be decomposed in terms of multiply-controlled SWAP gates performing a permutation of states, a multi-controlled single-excitation gate, and a reversal of the permutation, according to some embodiments.

FIG. 6 is a set of diagrams that represents multi-controlled single-excitation gates (left) and their associated decompositions in terms of a control single-excitation gate using a cascade of Toffoli gates (right), according to some embodiments.

FIG. 7 is a diagram of a controlled-controlled-SWAP gate and its decomposition in terms of three Fredkin gates with an auxiliary qubit, according to some embodiments.

FIG. 8 is a diagram of a quantum circuit for preparing the state c₁|110000 [symbol] +c₂|001100 [symbol] +c₃|000011 [symbol] +c₄|100100 [symbol] for arbitrary values of the coefficients c₁, c₂, c₃, c₄, according to some embodiments.

FIG. 9 is a set of diagrams that shows two different example architectures for variational circuits constructed from Givens rotations as fundamental building blocks, according to some embodiments.

FIG. 10A is a diagram showing a strategy for building quantum circuits for quantum chemistry, in which all single and double excitation gates that do not flip the spin of the particles are selected, according to some embodiments.

FIG. 10B is a diagram showing an adaptive strategy for building quantum circuits for quantum chemistry, in which, after initializing gate parameters, a gradient is computed for each gate and only those above a predefined threshold are retained, according to some embodiments.

FIG. 11 is a diagram showing a decomposition of a single excitation gate (left) into single-qubit rotations and CNOTs, according to some embodiments.

FIG. 12 is a diagram showing a decomposition of a double excitation gate into single-qubit rotations and CNOTs, according to some embodiments.

FIGS. 13-16 are flow diagrams showing example quantum chemistry methods, according to some embodiments.

FIG. 17 shows an example system for performing quantum chemistry methods of the present disclosure, in accordance with to some embodiments.

### DETAILED DESCRIPTION

Embodiments set forth herein include systems and methods for performing quantum chemistry simulations and calculations (e.g., predicting chemical and/or physical properties of molecules and materials) using “universal” quantum gates and/or circuits to approximate one or more particle-conserving unitaries. As used herein, a “universal” quantum gate (or set of gates) is a quantum gate to which any operation on a quantum computer can be reduced (e.g., a “building block” that can be used to approximate any unitary matrix arbitrarily well). Such universal quantum gates can also be referred to as “primitives.”

Universal gate sets for quantum computing have been known for decades, however, no universal gate set has previously been proposed for particle-conserving unitaries. Particle-conserving unitaries are operations of particular interest in quantum chemistry. In addition, some known quantum circuit architectures have been proposed to prepare quantum states for quantum chemistry, for example, in the context of variational quantum algorithms. Examples include chemically-inspired circuits, adaptive circuits, and hardware-efficient circuits. As such, those performing quantum chemistry are typically faced with choosing from among several variants of pre-defined quantum circuit architectures, rather than having access to universal tools for designing arbitrary algorithms. As described herein, in accordance with some embodiments, controlled single-excitation gates in the form of Givens rotations can be universal for particle-conserving unitaries. For example, a single-excitation gate, as discussed herein, can be used to describe an arbitrary U(2) rotation on the two-qubit subspace spanned by the states |01), |10), while leaving other states unchanged—a transformation that is analogous to a single-qubit rotation on a dual-rail qubit. The proof set forth herein is constructive, meaning that the result also provides an explicit method for compiling arbitrary particle-conserving unitaries. Additionally, a method for using controlled single-excitation gates to prepare an arbitrary state of a fixed number of particles is described. In accordance with methods described herein, Givens rotations are shown to be powerful and versatile primitives for designing variational circuits for quantum chemistry. One or more embodiments provide a unifying framework for quantum computational chemistry in which every algorithm is a unique recipe built from the same universal ingredients: Givens rotations.

Quantum algorithms for quantum chemistry often rely on the ability to prepare states that represent fermionic wavefunctions. These states can correspond, for example, to ground states and excited states of molecular Hamiltonians, which can then be used to compute properties of the molecule. For quantum chemistry systems that include a fixed number of particles on a given number of orbitals, valid quantum states occupy only a subspace of the available Hilbert space. Notably, in the Jordan-Wigner representation, the space of states with k particles in n spin-orbitals is spanned by the set of all n-qubit states with Hamming weight k, i.e., states with k ones and n−k zeros. To ensure that output states remain valid, quantum circuits for quantum chemistry should therefore preserve the number of particles.

Known universal gate sets capable of synthesizing arbitrary unitary operations include arbitrary single-qubit rotations and controlled NOT (CNOT) gates. No universal gate set, however, has previously been proposed specifically for particle-conserving unitaries, which as noted above are precisely the operations of interest in quantum chemistry. A universal set of particle-conserving gates as described herein provide a flexible and composable (i.e., user-designable) framework for designing arbitrary quantum circuits for quantum chemistry.

A constructive proof that controlled single-excitation gates are a universal gate set for particle-conserving unitaries is set forth herein. A single-excitation gate performs an arbitrary U(2) transformation in the subspace |01 [symbol] , |10 [symbol] while leaving other basis states unchanged:

$$( \begin{matrix}1 & 0 & 0 & 0 \\ 0 & a & c & 0 \\ 0 & b & d & 0 \\ 0 & 0 & 0 & 1\end{matrix} ) \qquad ( 1 )$$

where

$$U = ( \begin{matrix}a & c \\ b & d\end{matrix} )$$

is a general 2×2 unitary. Single excitation gates can be viewed as an extension of Givens rotations to unitary two-dimensional transformations. A controlled single-excitation gate, which applies this Givens rotation depending on the state of a third qubit, can be described by the unitary:

$$( \begin{matrix}1 & 0 \\ 0 & 1 \\ 0 & 0 \\ 0 & 0 \\ 0 & 0 \\ 0 & 0 \\ 0 & 0 \\ 0 & 0\end{matrix} \begin{matrix}0 \\ 0 \\ 1 \\ 0 \\ 0 \\ 0 \\ 0 \\ 0\end{matrix} \begin{matrix}0 \\ 0 \\ 0 \\ 1 \\ 0 \\ 0 \\ 0 \\ 0\end{matrix} \begin{matrix}0 \\ 0 \\ 0 \\ 0 \\ 1 \\ 0 \\ 0 \\ 0\end{matrix} \begin{matrix}0 \\ 0 \\ 0 \\ 0 \\ 0 \\ a \\ b \\ 0\end{matrix} \begin{matrix}0 \\ 0 \\ 0 \\ 0 \\ 0 \\ c \\ d \\ 0\end{matrix} \begin{matrix}0 \\ 0 \\ 0 \\ 0 \\ 0 \\ 0 \\ 0 \\ 1\end{matrix} ) \qquad ( 2 )$$

In addition to the universality result, an explicit algorithm is described herein that uses excitation gates to prepare an arbitrary state with a fixed number of particles. Analytical gradient formulas for Givens rotations are derived and shown herein to be ideal building blocks in variational quantum circuits for quantum chemistry.

Particle-Conserving Unitaries

In the description that follows, particles and operations are described without explicit reference to electrons, spin-orbitals, fermionic operators, etc., for clarity in explanation, however embodiments of the present disclosure are applicable to the modeling and prediction of quantum chemistry systems that include electrons, spin-orbitals, fermionic operators, etc. Define the qubit ladder operators

$${σ}^{†} = \frac{X + i Y}{2} , σ = \frac{X + i Y}{2} ,$$

where X, Y are Pauli matrices, and the total number operator “N” is:

N=Σᵢσᵢ^{†}σᵢ. (3)

For a computational basis state |x), it holds that N=|x [symbol] =w(x) |x [symbol] , where w(x) is the Hamming weight of the bit string x, which can be defined as equal to the number of particles. Eigenstates of the total number operator are defined herein as states having a fixed number of particles. A unitary gate U is deemed particle-conserving if:

[U,N]=0. (4)

A particle-conserving unitary maps states with a fixed number of particles to other states with a fixed number of particles. Any product of particle-conserving unitaries U, V is also particle-conserving, so any quantum circuit consisting of particle-conserving quantum gates (i.e., including only particle-conserving quantum gates and not any non-particle-conserving quantum gates) is guaranteed to perform a particle-conserving transformation.

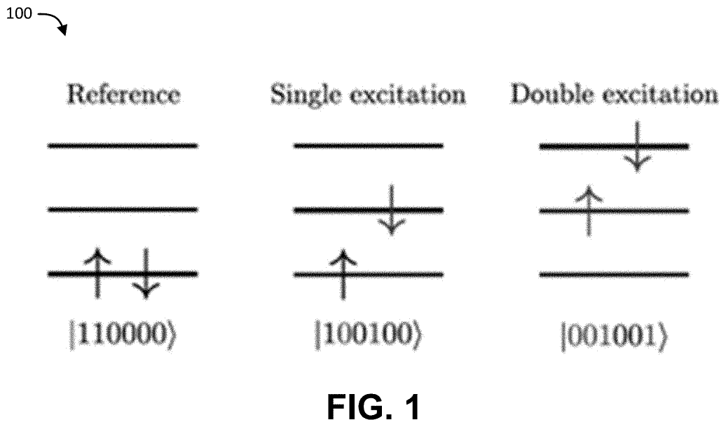

The space of all states with k particles on n qubits, denoted as [symbol] ₖ, is spanned by the set of computational basis states with Hamming weight k. These states can be partitioned in terms of permutations from a reference state, and indeed any state of a fixed number of particles can be viewed/interpreted as an excitation from a reference state. Herein, unless stated otherwise, the state |11 . . . 100 . . . 0 [symbol] with all particles in the first k qubits is considered the reference state. This reference state is shown in FIG. 1 (discussed below). The Hamming distance Σᵢ₌₁ⁿXᵢ ⊕ [symbol] ᵢ can be used to denote the number of qubits where the computational basis states 1x) and 1y) differ. For states with an equal number of particles, the Hamming distance is an even number.

Two states |X [symbol] and | [symbol] [symbol] of an equal number of particles are said to differ by an excitation of order [symbol] if their Hamming distance is equal to 2 [symbol] . For example, the states |1100 [symbol] and |0101 [symbol] differ by a single excitation (order 1) from the first to the fourth qubit. Similarly, the state |0011 [symbol] differs from |1100 [symbol] by a double excitation (order 2).

FIG. 1 shows an example Jordan-Wigner representation of states with a fixed number of particles. Each qubit corresponds to an orbital with a specific spin orientation. The state of the qubit determines whether that spin-orbital is occupied or not. All basis states can be obtained from a reference state, in this case |110000 [symbol] , by a specific excitation. For example, the state |100100 [symbol] is obtained by exciting a particle from qubit 2 to qubit 4, while the state |001001 [symbol] is obtained by exciting both particles to qubits 3 and 4.

Any particle-conserving unitary acting on states with k particles on n qubits can be represented as a block-diagonal unitary performing a general U(d) transformation on the subspace [symbol] ₖ, with dimension d=dim( [symbol] ₖ). For universality, it is therefore sufficient to consider a set of particle-conserving gates that is universal for the subspace [symbol] ₖ.

### Givens Rotations

As discussed above, any two states with a fixed number of particles differ by an excitation of a given order. It is therefore convenient to work with a set of quantum gates that create superpositions between the original and the excited state. In a straightforward non-trivial case of a single particle and two qubits, these correspond to gates that perform arbitrary U(2) rotations between the two states |10 [symbol] , |01 [symbol] , while leaving other basis states unchanged. For example, restricting to the case where the gate has only real parameters, a two-qubit particle-conserving unitary can be written as:

$$G ( θ ) = ( \begin{matrix}1 & 0 & 0 & 0 \\ 0 & cos ( θ ) & - s in ( θ ) & 0 \\ 0 & sin ( θ ) & cos ( θ ) & 0 \\ 0 & 0 & 0 & 1\end{matrix} ) \qquad ( 5 )$$

where the ordering |00 [symbol] , |01 [symbol] , |10 [symbol] , |11 [symbol] of two-qubit computational basis states is used.

This is an example of a Givens rotation: a rotation in a two-dimensional subspace of a larger space. In this case, the Givens rotation acts as a two-qubit single-excitation gate, coupling states that differ by a single excitation. More generally, the concept of Givens rotations can be extended to U(2) transformations in two-dimensional subspaces, where a general single-excitation gate can be written as:

$$G = ( \begin{matrix}1 & 0 & 0 & 0 \\ 0 & a & c & 0 \\ 0 & b & d & 0 \\ 0 & 0 & 0 & 1\end{matrix} ) , \qquad ( 6 )$$

where |a|²+|c|²=|b|²+|d|²=1 and ab*+bd*=0 to ensure unitarity. Here, the ordering |00 [symbol] , |01 [symbol] , |10 [symbol] , |11 [symbol] of two-qubit computational basis states is used. Four-qubit double-excitation gates G⁽²⁾ can also be considered, which perform a general U(2) rotation on the subspace spanned by the states |0011 [symbol] , |1100 [symbol] :

G⁽²⁾|0011 [symbol] =a|0011 [symbol] +b|1100 [symbol] , (7)

G⁽²⁾|1100 [symbol] =c|1100 [symbol] +d|0011 [symbol] , (8)

while leaving all remaining four-qubit states unchanged. Double-excitation gates can also perform rotations in two-dimensional subspaces defined by pairs of four-qubit states with Hamming distance four, namely |1010 [symbol] , |0101 [symbol] and |1001 [symbol] , |0110 [symbol] .

The excitation gates of order [symbol] can then be generalized. These are unitary Givens rotations acting on the space of 2 [symbol] qubits that couple the states | [symbol] :=|1 [symbol] |0 [symbol] and | [symbol] :=|0 [symbol] |1 [symbol] as:

[symbol]

|0 [symbol] 1 [symbol] =a|0 [symbol] 1 [symbol] [symbol] +b|1 [symbol] 0 [symbol] [symbol] , (9)

[symbol]

|1 [symbol] 0 [symbol] =c|1 [symbol] 0 [symbol] [symbol] +d|0 [symbol] 1 [symbol] [symbol] , (10)

while acting as the identity on all other states. Similar Givens rotations can be defined for permutations of the states |1 [symbol] 0 [symbol] [symbol] , 0 [symbol] 1 [symbol] [symbol] , i.e., excitation gates of order [symbol] also include rotations on all pairs of states of 2 [symbol] qubits with Hamming distance 2 [symbol] . By construction, these excitation gates are particle-conserving since they only couple states having the same number of particles.

Next, consider controlled excitation gates, which apply an excitation gate depending on the state of a control qubit. In particular, consider the three-qubit controlled single-excitation gate:

$$CG = ( \begin{matrix}1 & 0 \\ 0 & 1 \\ 0 & 0 \\ 0 & 0 \\ 0 & 0 \\ 0 & 0 \\ 0 & 0 \\ 0 & 0\end{matrix} \begin{matrix}0 \\ 0 \\ 1 \\ 0 \\ 0 \\ 0 \\ 0 \\ 0\end{matrix} \begin{matrix}0 \\ 0 \\ 0 \\ 1 \\ 0 \\ 0 \\ 0 \\ 0\end{matrix} \begin{matrix}0 \\ 0 \\ 0 \\ 0 \\ 1 \\ 0 \\ 0 \\ 0\end{matrix} \begin{matrix}0 \\ 0 \\ 0 \\ 0 \\ 0 \\ a \\ b \\ 0\end{matrix} \begin{matrix}0 \\ 0 \\ 0 \\ 0 \\ 0 \\ c \\ d \\ 0\end{matrix} \begin{matrix}0 \\ 0 \\ 0 \\ 0 \\ 0 \\ 0 \\ 0 \\ 1\end{matrix} ) \qquad ( 11 )$$

A particular example of a controlled single-excitation gate is the controlled SWAP, or Fredkin gate:

$$F = ( \begin{matrix}1 & 0 \\ 0 & 1 \\ 0 & 0 \\ 0 & 0 \\ 0 & 0 \\ 0 & 0 \\ 0 & 0 \\ 0 & 0\end{matrix} \begin{matrix}0 \\ 0 \\ 1 \\ 0 \\ 0 \\ 0 \\ 0 \\ 0\end{matrix} \begin{matrix}0 \\ 0 \\ 0 \\ 1 \\ 0 \\ 0 \\ 0 \\ 0\end{matrix} \begin{matrix}0 \\ 0 \\ 0 \\ 0 \\ 1 \\ 0 \\ 0 \\ 0\end{matrix} \begin{matrix}0 \\ 0 \\ 0 \\ 0 \\ 0 \\ 0 \\ 1 \\ 0\end{matrix} \begin{matrix}0 \\ 0 \\ 0 \\ 0 \\ 0 \\ 1 \\ 0 \\ 0\end{matrix} \begin{matrix}0 \\ 0 \\ 0 \\ 0 \\ 0 \\ 0 \\ 0 \\ 1\end{matrix} ) \qquad ( 12 )$$

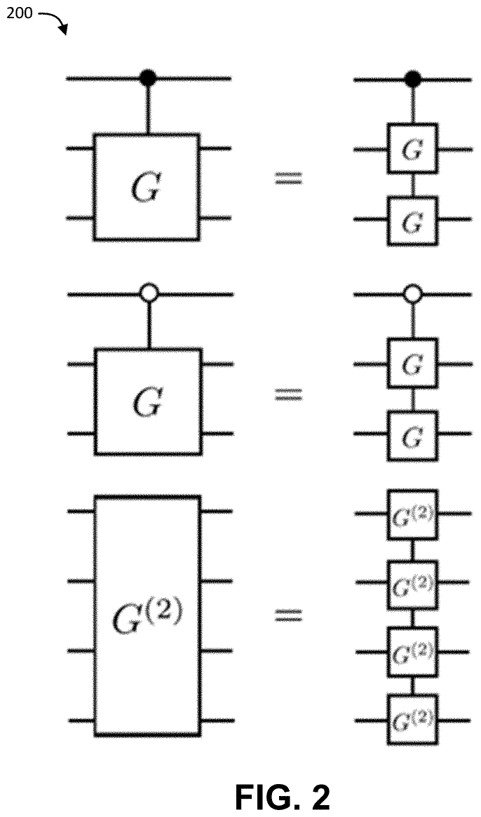

The phrase “controlled gates” typically refers to the case where a gate is applied only if the control qubit is in state |1 [symbol] . As used herein, the phrase “controlled gate” is defined as also including the case when gates are applied only if the control qubit is in state |0 [symbol] . All such controlled gates are also particle-conserving. Controlled single-excitation gates and double-excitation gates are illustrated in FIG. 2.

FIG. 2 shows an example three-qubit controlled single-excitation gate “G,” which is universal for particle-conserving unitaries, and a four qubit double-excitation gate “G⁽²⁾.” The controlled single-excitation gate on the top denotes a control on state |1 [symbol] , while the controlled single-excitation gate below it shows a control on state |0 [symbol] . The righthand side of FIG. 2 shows alternative representations of the excitation gates, which can be useful for drawings circuits when the qubits are not adjacent. The bottom circuit in FIG. 2 is a double-excitation gate.

### Proof of Universality

In this section, controlled single-excitation gates are shown to be universal for particle-conserving unitaries. The universality proof includes the following steps:
1. For particle-conserving unitaries, relevant U(2) transformations are shown to be excitation gates controlled on multiple qubits. Given that U(d) transformations can be decomposed into products of U(2) transformations, it follows that excitation gates controlled on multiple qubits are universal for particle-conserving unitaries.
2. Any excitation gate (e.g., an excitation gate of arbitrary order) controlled on multiple qubits is shown to be decomposable in terms of multiply-controlled single-excitation gates.
3. Single excitation gates controlled on multiple qubits are shown to be decomposable into three-qubit controlled single-excitation gates, which are therefore also universal.

The foregoing proof shows that single-excitation gates as described herein can be used in a manner analogous to that of single-qubit gates. Indeed, the subspace |10 [symbol] , |01 [symbol] can be interpreted as a dual-rail encoding of a single qubit. Fredkin gates, which are a specific type of control single-excitation gate, can be used to extend controlled single-excitation gates to controlled gates over multiple qubits. Friedkin gates can be universal, for example, for reversible computations in dual-rail encodings.

Excitation Gates with Multiple Controls

A U(d) transformation can be decomposed into a product of U(2) transformations acting on arbitrary two-dimensional subspaces. As discussed above, any state of a fixed number of particles can be obtained by applying an excitation to a reference state. This result holds more generally: any two states of a fixed number of particles differ by a specific excitation.

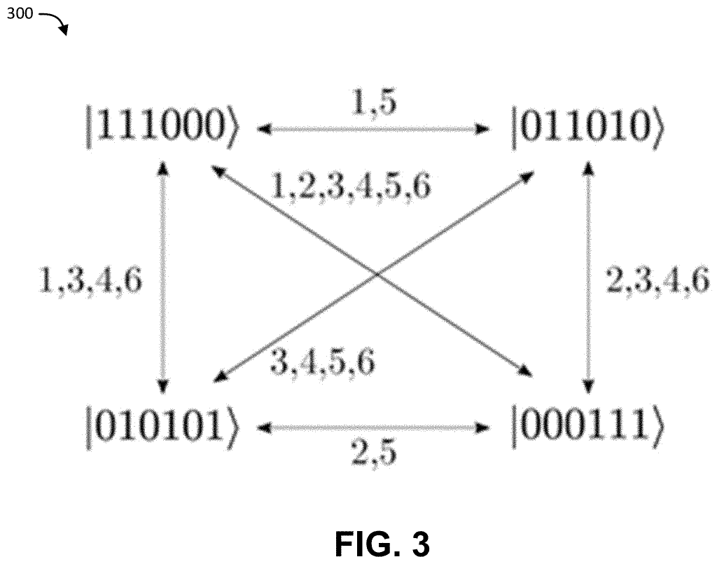

FIG. 3 shows that any pair of states of a fixed number of particles can be related by an excitation. For example, the states |111000 [symbol] and |011010 [symbol] differ by a single excitation between qubits 1 and 5. The states |011010 [symbol] and |000111) differ by a double excitation between qubits 2,3,4,6, while |000111 [symbol] and |111000 [symbol] are connected by a triple excitation acting on all qubits 1, 2, 3, 4, 5, 6.

Consider two k-particle states |x [symbol] , |y [symbol] on n qubits, with Hamming distance 2 [symbol] . Without loss of generality, since this can be ensured by relabeling, suppose the first k− [symbol] qubits are set to 1 for both states and the last n−k− [symbol] qubits are set to 0. For the remaining 2 [symbol] qubits, the value is different for both states, meaning that they can be mapped to each other by exciting the particles from the [symbol] occupied qubits to the [symbol] unocuppied qubits. For example, the states |111000 [symbol] and |110010 [symbol] , which have Hamming distance 2, differ by a single excitation from qubit 3 to qubit 5. Similarly, the states |011010 [symbol] and |010101 [symbol] differ by a double excitation from qubits 3 and 5 to qubits 4 and 6. This connection between states and excitations is illustrated in FIG. 3.

A U(2) rotation in the subspace spanned by the k-particle states |x [symbol] , |y [symbol] is therefore equivalent to a unitary performing the transformation:

U|x [symbol] =a|x [symbol] + [symbol] [symbol] [symbol] (13)

U| [symbol] [symbol] =c| [symbol] [symbol] + [symbol] |x [symbol] (14)

while leaving every other basis state unchanged. For the specific case of states with n=2 [symbol] qubits, this is accomplished by a unitary Givens rotation [symbol] as in Eqs. (9) and (10). However, when applied to states with n>2 [symbol] qubits, the gate [symbol] acts non-trivially on any states where 2 [symbol] qubits are set to |x [symbol] or |y [symbol] , regardless of the state of the remaining qubits. For example, if |z) is a basis state of m=n−2 [symbol] qubits, it holds that:

[symbol]

|z [symbol] |x [symbol] =a|z [symbol] |x [symbol] +b|z [symbol] | [symbol] [symbol] (15)

for all z. To address this issue, [symbol] can be applied, controlled on the state of the remaining n−2 [symbol] qubits. The notation C(m) [symbol] can be used to denote a [symbol] gate controlled in the state of m qubits. Controlling on the remaining qubits being on state |z* [symbol] and defining |x′ [symbol] =|z* [symbol] |x [symbol] and |y′ [symbol] =|z* [symbol] |y [symbol] , it follows that:

C^{(m)} [symbol] |x′ [symbol] =a|x′ [symbol] +b|y′ [symbol] , (16)

C^{(m)} [symbol] |y′ [symbol] =c|y′ [symbol] +b|x′ [symbol] , (17)

while leaving all other basis states unchanged. This is the desired two-dimensional transformation.

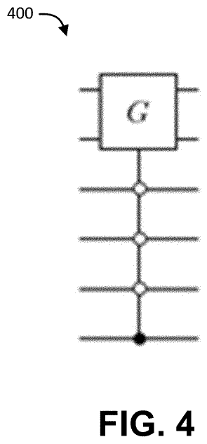

For example, consider the states |100011 [symbol] and |010011 [symbol] , which differ by an excitation from the first qubit to the second qubit, and coincide on the remaining four qubits. A non-controlled single-excitation gate acting on the first two qubits would also perform a transformation on other subspaces, for example the subspace spanned by |101101 [symbol] and |011100 [symbol] . Controlling on the last four qubits being in state |0011 [symbol] , however, ensures that the gate C⁽⁴⁾G acts non-trivially only on the target two-dimensional subspace. The role of multiple controls is exemplified in FIG. 4.

C⁽⁴⁾G|100001 [symbol] =a|100001 [symbol] +b|010001

[symbol]

C⁽⁴⁾G|010010 [symbol] =|010010

[symbol]

FIG. 4 shows that a single-excitation gate G can act non-trivially on many states. To ensure that the desired rotation happens only between two target states, a multi-controlled gate C⁽⁴⁾G can be applied that acts as the identity on non-target states. In this example, the goal is to perform a rotation in the subspace of states |100001 [symbol] , |010001 [symbol] . This can be achieved by applying a single-excitation gate to the first two qubits, controlled on the state of the last four being |0001). This ensures that other states, such as |010010 [symbol] , are left unchanged. A circuit diagram of this multi-controlled single-excitation gate is shown in FIG. 4.

It is observed that any two-level U(2) unitary Givens rotation on the subspace of k-particle states on n qubits can be implemented in terms of multiple-control excitation gates C^{(m)} [symbol] , where n=2 [symbol] +m. This implies that multiple-control excitation gates are universal for particle-conserving operations.

Single-Excitation Gates with Multiple Controls

Multiple-control excitation gates can be decomposed in terms of multiple-control single-excitation gates C^{(m)}G. Suppose it is desired to decompose a U(2) Givens rotation on the subspace spanned by |x [symbol] , |y [symbol] , where the states have Hamming distance 2 [symbol] . The goal is to employ single-excitation gates to perform a permutation of all basis states such that the permuted versions of |x [symbol] and |y [symbol] differ by a single excitation. This can be achieved following principles that are similar to the construction of Gray codes, as shown below. A controlled single excitation gate can then be applied to the basis states, followed by a reversal of the permutation.

For example, the states |101001 [symbol] and |010110 [symbol] , which differ by a triple excitation, can be linked through the sequence |101001 [symbol] →|011001 [symbol] →|010101 [symbol] →|010110 [symbol] , where each new state differs from the previous one by a single excitation. Each of the states in this sequence can be obtained from the previous one by applying a SWAP gate:

$$SWAP = ( \begin{matrix}1 & 0 & 0 & 0 \\ 0 & 0 & 1 & 0 \\ 0 & 1 & 0 & 0 \\ 0 & 0 & 0 & 1\end{matrix} ) \qquad ( 18 )$$

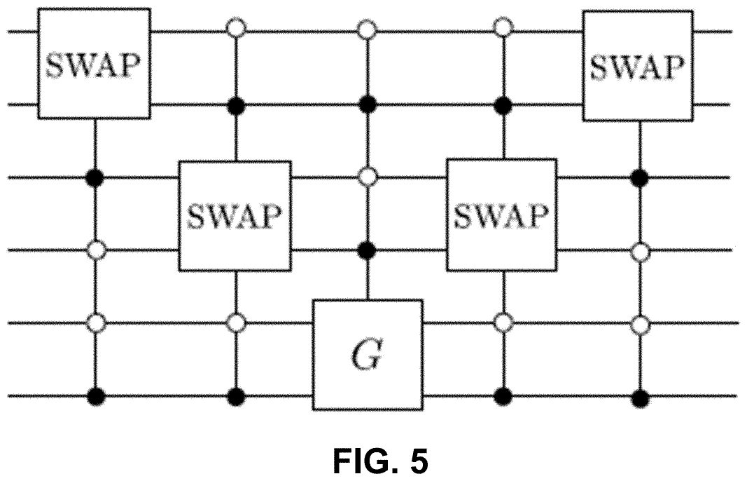

controlled on the state of all remaining qubits, i.e., by applying a C^{(m)}SWAP gate. The SWAP gate is a special case of a single-excitation gate that swaps the state of one qubit with the state of another qubit. As before, the control is used to ensure that the resulting permutation occurs non-trivially only on the two-dimensional target subspace. This procedure is illustrated in FIG. 5.

The foregoing method is now described in greater detail. Without loss of generality, suppose that the states |x [symbol] and |y [symbol] differ on the first 2 [symbol] qubits. A first step is to outline an ordered sequence of computational basis states |g₁ [symbol] , |g₂ [symbol] , . . . , | [symbol] such that all |gᵢ [symbol] , |gᵢ₊₁ [symbol] differ by a single excitation and where |x [symbol] =|g₁ [symbol] and |y [symbol] =| [symbol] . To build the circuit implementing the decomposition, the following steps may be performed:
1. Apply a SWAP gate to the qubits where |x [symbol] =|g₁ [symbol] and |g₂ [symbol] differ, controlled on all other qubits. This has the effect of swapping |g₁ [symbol] , |g₂), while leaving all other states unchanged.
2. Follow the same procedure to swap |g₂ [symbol] , |g₃ [symbol] , then |g₃ [symbol] , |g₄ [symbol] , and all other states until the final swap between | [symbol] , | [symbol] . This sequence of operations has the effect of mapping |x [symbol] →| [symbol] while | [symbol] =|y [symbol] is left unchanged.
3. Since | [symbol] , | [symbol] differ by a single excitation, a multi-controlled single-excitation gate that acts only on the subspace spanned by these states is performed.
4. The circuit is completed by reverting all the swaps such that the resulting transformation is a rotation in the sub-space spanned by |x [symbol] and |y [symbol] , as desired.

FIG. 5 shows an example method for connecting any pair of states by a sequence of particle-conserving multi-controlled SWAP gates. The initial state |x [symbol] =|101001), shown below, differs from the target state |010110) by a triple excitation, which can be decomposed in terms of single excitations using a sequence of intermediary states. A circuit implementing a triple-excitation rotation can then be decomposed in terms of (i) multi-controlled SWAP gates performing a permutation of states, (ii) a multi-controlled single-excitation gate, and (iii) a reversal of the permutation.

|g₁ [symbol] =|x [symbol] =|101001

[symbol]

C⁽⁴⁾SWAP|g₁ [symbol] =|011001 [symbol] =|g₂

[symbol]

C⁽⁴⁾SWAP|g₂ [symbol] =|010101 [symbol] =|g₃

[symbol]

C⁽⁴⁾G|g₃ [symbol] =|010110 [symbol] =|g₄ [symbol] =|y

[symbol]

### C. Controlled Single-Excitation Gates are Universal

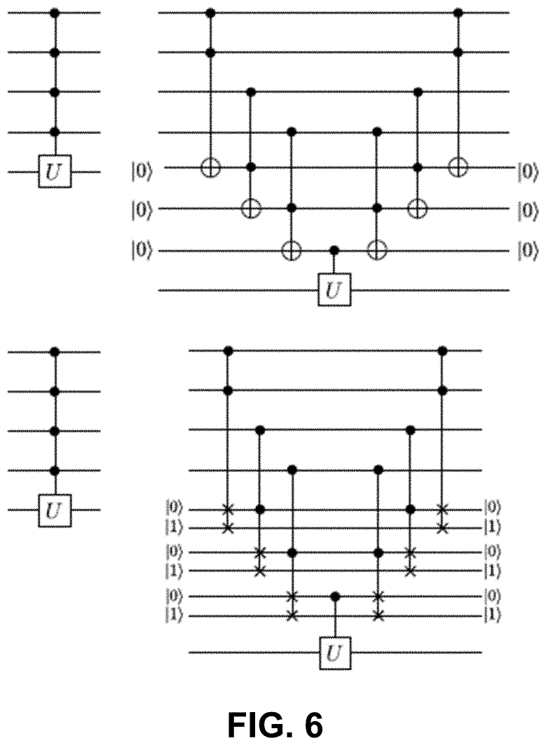

Given a quantum gate controlled on a single qubit, a variety of known methods exist for extending the control to additional qubits. Suppose one desires to control the operation on the state of m qubits |z₁z₂ . . . zₘ [symbol] . The strategy relies on employing [symbol] −1 auxiliary qubits and Toffoli gates (control CNOT gates), as shown in FIG. 6.

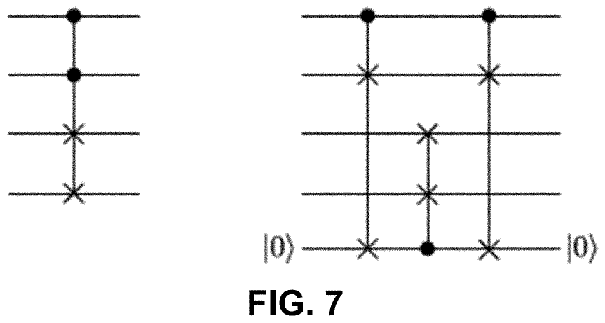

Toffoli gates are not particle-conserving, but they can be decomposed in terms of particle-conserving Fredkin gates by replacing the auxiliary qubits with dual-rail qubits |\tilde{0} [symbol] :=|01 [symbol] ,|\tilde{1} [symbol] :=|10 [symbol] . In this case a Toffoli gate is equivalent to a controlled-controlled-SWAP gate, since swapping |01 [symbol] and |10 [symbol] applies a NOT gate to the dual-rail qubit. The controlled-controlled-SWAP gate can then be decomposed into three Fredkin (controlled-SWAP) gates with the help of an auxiliary qubit, as shown in FIG. 7. For simplicity, consider the case where gates are applied when the control qubit is in state |1 [symbol] , which can be generalized to controlling on arbitrary values.

FIG. 6 shows that a multi-controlled single-excitation gate (left) can be decomposed in terms of a controlled single-excitation gate using a cascade of Toffoli gates (right). The Toffoli gates can be decomposed in terms of the particle-conserving controlled-controlled-SWAP gate using additional dual-rail qubits.

As discussed above, single-excitation gates controlled on multiple qubits can be universal for particle-conserving unitaries, and can be decomposed into controlled single-excitation gates, which are therefore also universal.

### State Preparation

Universal gate sets for particle-conserving unitaries can also be used to prepare arbitrary states of a fixed number of particles. Multiply-controlled excitation gates can be used for this purpose.

Consider a system of k particles on n qubits, spanning a space of dimension

$$d = ( \begin{matrix}n \\ k\end{matrix} ) .$$

Any such state can be written as |ψ [symbol] =Eₓcₓ|x [symbol] , where the sum is over all n-bit strings x of Hamming weight k. As shown above, an arbitrary U(2) rotation in the subspace of any pair of states |x [symbol] , |y [symbol] can be performed by a suitable decomposition into controlled single-excitation gates.

Consider a lexicographical labeling of all bit strings of Hamming weight k as |x₁ [symbol] , |x₂ [symbol] , . . . , |x_{d} [symbol] , where

$$d = ( \begin{matrix}n \\ k\end{matrix} ) .$$

For example, in the case of [symbol] =3 and k=2, one obtains |x₁ [symbol] =|011 [symbol] , |x₂ [symbol] =|101 [symbol] , and |x₃ [symbol] =|110 [symbol] . An arbitrary state can then be written as:

|ψ [symbol] =Σᵢ₋₁^{d}cᵢ|xᵢ [symbol] . (19)

A method to prepare any such state starting from the reference state |x₁ [symbol] , in accordance with some embodiments, is as follows. First, the multi-controlled excitation operation in the subspace |x₁ [symbol] , |x₂ [symbol] is applied, which performs the mapping:

|x₁ [symbol] →α₁|x₁ [symbol] +c₂|x₂ [symbol] . (20)

where α₁=\sqrt{1−|c₂|²}. Then, the multi-controlled excitation operation is applied in the subspace |x₁ [symbol] , |x₃ [symbol] . This performs the mapping:

a₁|x₁ [symbol] +c₂|x₂ [symbol] →α₁α₂|x₁ [symbol] +c₂|x₂ [symbol] +α₁c′₃|x₃ [symbol] (21)

where c′₃=c₃/α₁ and α₂=\sqrt{1−c′₃|²}. This ensures that the coefficient in front of |x₃ [symbol] is precisely the desired one, c₃. This process can be repeated for each of the remaining states |x₄), . . . , |x_{d}). The result is to prepare the state:

$$\vert ψ 〉 = a \vert {x}_{1} 〉 + {\sum }_{i - 2}^{d} {c}_{i} \vert {x}_{i} 〉 , \qquad ( 22 )$$

where α=Πᵢ₌₁^{d-1} αᵢ. This state is normalized, which from Eq. (19) implies that |α|²=|c₁|². To ensure that, in fact, α=c₁=|c₁|e^{1θ}, it suffices to choose α₁, α₂, . . . , α_{d-2} to be positive real numbers, and set α_{d-1}=|α_{d-1}|e^{iθ} to prepare the desired state. Note that for a unitary

$$U = ( \begin{matrix}a & b \\ c & d\end{matrix} ) ,$$

a can be guaranteed to be real for any c by choosing:

$$\begin{matrix}d = \sqrt{1 - {\vert {c}_{2} \vert}^{2}} \\ {b}^{*} = \frac{c \sqrt{1 - {\vert {c}_{2} \vert}^{2}}}{a} .\end{matrix}$$

The present strategy can be customized for particular cases. Since the gates act only on a specific superposition of basis states, controls may only be applied on qubits where the states in the superposition differ. This is useful, for example, if the target state has support only on a specific subspace. For example, the first excitation gate performing the mapping |xᵢ [symbol] →α₁|x₁ [symbol] +c₂|x₂ [symbol] may not need to be controlled. Furthermore, excitation gates can be chosen to act different reference states to create new superpositions.

Consider the six-qubit state:

c₁|110000 [symbol] +c₂|001100 [symbol] +c₃|000011 [symbol] +c₄|100100 [symbol] , (23)

which corresponds to a superposition of the four basis states that contribute most significantly to the ground-state energy of the H₃⁺ molecule in a minimal basis set. The state can be prepared as follows.

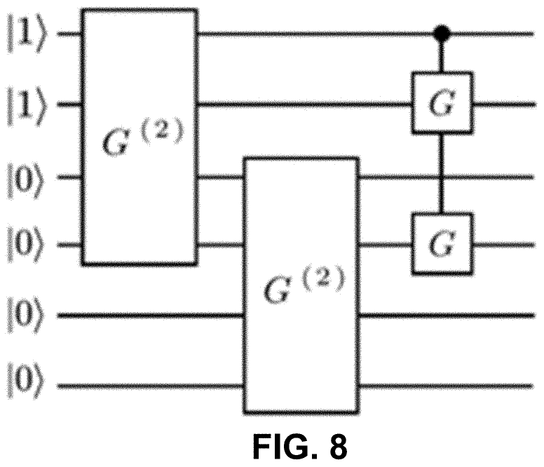

Starting from the state |110000 [symbol] , apply a double-excitation gate to the first four qubits to prepare the state a₁|110000 [symbol] +c₂|001100 [symbol] . This need not be controlled on any qubit. Then, apply a double-excitation gate to qubits 3, 4, 5, and 6 to prepare the state a₁|110000 [symbol] +c₂|001100 [symbol] +c₃|000011 [symbol] , where |001100 [symbol] is used as the reference. This, again, need not be controlled on any qubit. To obtain the desired state, apply a single-excitation gate to qubits 2 and 4, controlled on the first qubit being in state 11), which prevents mixing with the state |001100 [symbol] . This construction is shown in FIG. 8.

FIG. 8 shows an example quantum circuit for preparing the state c₁|110000 [symbol] +c₂|001100 [symbol] +c₃|000011 [symbol] +c₄|100100 [symbol] for arbitrary values of the coefficients c₁, c₂, c₃, c₄. Double-excitation gates G⁽²⁾ are applied to the first four qubits and then to the last four qubits. A controlled single-excitation gate is then applied to qubits 2 and 4, controlled on the state of the first qubit.

### Variational Quantum Circuits

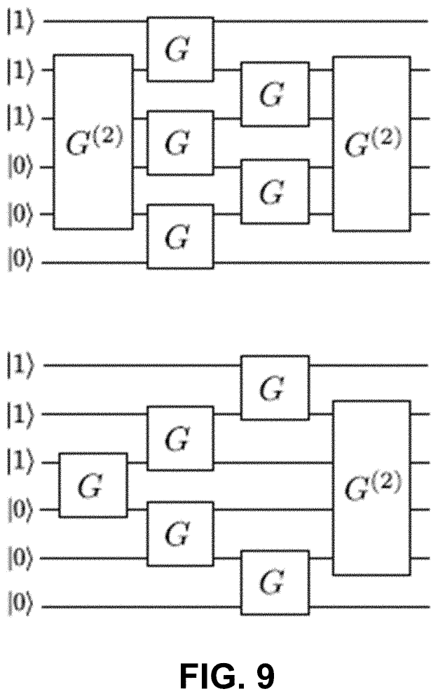

In some embodiments, variational quantum circuits may be constructed using the controlled single-excitation gates described herein as building blocks. For example, the state preparation algorithm described above could be employed as a template, where the rotation angles for each gate are free parameters of the model. In this context, multiple controls may not be used. Rather, uncontrolled excitation gates may be used, and a larger subspace of states may be reached using fewer gates. For example, in the example of FIG. 8, dropping the control on the last single excitation gate would lead to a state with a non-zero coefficient on the additional basis state |010100 [symbol] . Examples of how to design variational circuits using Givens rotations as building blocks are shown in FIG. 9.

FIG. 9 shows two different circuit architectures constructed using particle-conserving operations (e.g., Givens rotations as described herein) as the fundamental building blocks. Since these operations are particle-conserving, it is possible to compose them arbitrarily to create various types of particle-conserving circuits. Excitation gates without controls can be used to access larger subspaces with fewer gates, whose parameters may then be optimized for specific purposes.

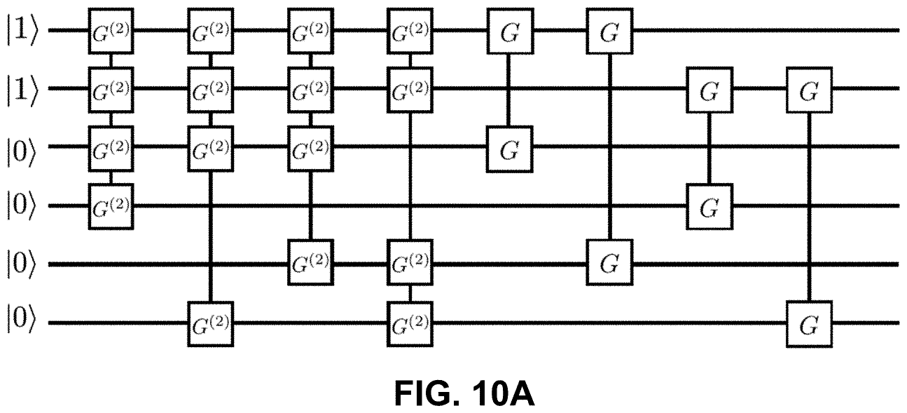

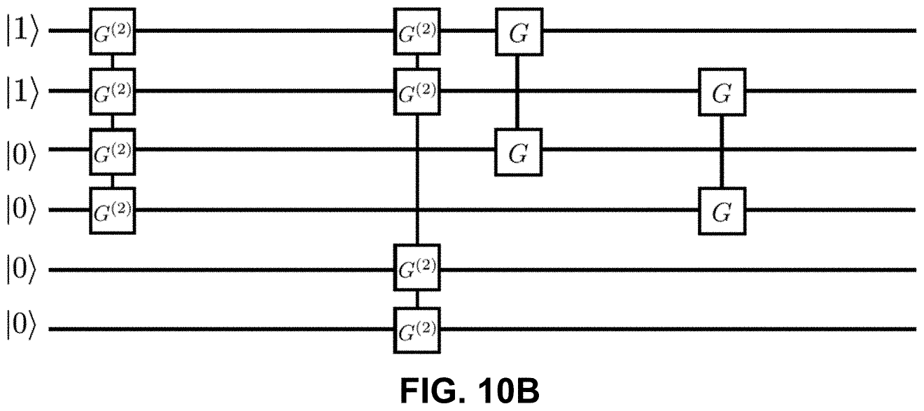

As an example, in the context of quantum computing, the unitary coupled-cluster singles and doubles (UCCSD) ansatz is often expressed in terms of fermionic operators, which are then mapped to complicated qubit gates. As an alternative, consider a UCCSD circuit where single excitations and double excitations are respectively implemented using Givens rotations G and G⁽²⁾. A quantum circuit can then be defined that includes all possible single excitation gates and double excitation gates that act non-trivially on the reference state without flipping the spin of the excited particles. The resulting circuit is analogous to a Trotterized implementation of UCCSD to first level, but where all gates are Givens rotations. This is illustrated, by way of example, in FIG. 10.

### Adaptive Circuits

Adaptive strategies such as those presented in Refs. [22, 23] can be implemented by selecting Givens rotations instead of fermionic excitations in the constructions. The main idea is that instead of designing circuits that work well for all molecules, we can instead build specific circuits that are custom-built for each molecule. Hence, what is general is the method for building custom circuits, not the circuits themselves.

A simple yet powerful strategy is to build a circuit that includes all relevant double excitation gates and single excitation gates, randomly initialize all parameters, and compute the gradient for each gate. The final circuit can then be constructed by retaining only those gates for which the norm of their gradient exceeds a fixed threshold. This is shown in FIGS. 10A-10B, discussed below.

### Analytical Gradients

The following describes a derivation of analytic gradient formulas for Givens rotations. If \tilde{H} is the generator of a unitary Ũ(θ)=e^{i\tilde{H}θ}, then the generator of the unitary U(θ)= [symbol] ⊕Ũ(θ) is H=0⊕\tilde{H}, where 0 denotes the zero operator. Generators of this form can be decomposed as follows:

H=½(H₊+H₋), (24)

H₊=(± [symbol] )⊕\tilde{H}. (25)

It can then be written that:

U(θ)=e^{iθG}+/²e^{iθG}−/², (26)

with the gates defined as:

U_{±}(θ)=e^{iθG±}. (27)

The operators H_{±} satisfy:

H_{±}²= [symbol] , (28)

H₊,H₋=0. (29)

FIG. 10A is a diagram showing a strategy for building quantum circuits for quantum chemistry, in which all single and double excitation gates that do not flip the spin of the particles are selected, according to some embodiments.

FIG. 10B is a diagram showing an adaptive strategy for building quantum circuits for quantum chemistry, in which, after initializing gate parameters, a gradient is computed for each gate and only those above a predefined threshold are retained, according to some embodiments.

It can be shown that any unitary U(θ) with a generator that is self-inverse satisfies the parameter-shift rule:

$$\frac{\partial C ( θ )}{\partial θ} = \frac{C ( θ + s ) - C ( θ - s )}{2 sin ( s )} , \qquad ( 30 )$$

for any cost function that can be written as C(θ)= [symbol] ψ|U†(θ)KU(σ)|ψ [symbol] , where K is an observable. This parameter-shift rule applies to the gates U_{±}(θ), which means that derivatives of U(θ) can be obtained by writing U(θ)=U₊(θ/2)U₋(θ/2) and computing derivatives of the gates U_{±}(θ). This technique can be employed for any Givens rotation whose generator \tilde{H} is self-inverse. For example, in the case of the Givens rotation of Eq. (5), one can write G₊=G₊(θ/2)G₋(θ/2), where the unitaries:

$${G}_{\pm } ( θ ) = ( \begin{matrix}{e}^{\pm i θ} & 0 & 0 & 0 \\ 0 & cos θ & - s in θ & 0 \\ 0 & sin θ & cos θ & 0 \\ 0 & 0 & 0 & {e}^{\pm i θ}\end{matrix} ) , \qquad ( 31 )$$

satisfy the parameter-shift rule.

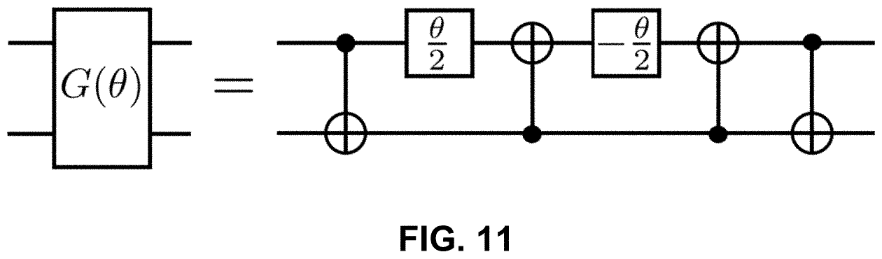

FIG. 11 is a diagram showing a decomposition of a single excitation gate (left) into single-qubit rotations and CNOTs, according to some embodiments. The gates denoted by ±σ/2 are Pauli Y rotations. The middle four gates constitute a controlled Y rotation.

### Decompositions of Excitation Operators

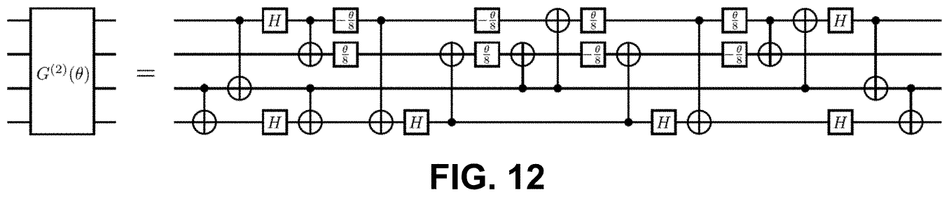

While controlled single excitation operators comprise a universal gate set, hardware implementations may involve a decomposition of these operators over a gate set of single-qubit rotations and CNOTs. FIG. 11, discussed above, presents such a decomposition for a single-excitation gate. Such a decomposition can be extended to a controlled version by applying a control to each individual gate, thereby producing Toffolis and controlled-Y rotations, each of which can be further decomposed over the gate set. FIG. 12 presents a decomposition for a four-qubit double-excitation gate into single-qubit rotations and CNOTs. All gates denoted by ±θ/8 are Pauli Y rotations.

Controlled single-excitation gates as described herein are universal for particle-conserving unitaries, which are the relevant operations for quantum chemistry. These three-qubit gates are unitary Givens rotations performing a transformation in a two-dimensional subspace of states |01 [symbol] , |10 [symbol] , controlled on the state of a third qubit. The states |01 [symbol] , |10 [symbol] can be interpreted as a dual-rail encoding of a single qubit, thus making controlled single-excitation gates analogous to controlled single-qubit gates.

The proof of universality discussed above presumes that excitation gates can be controlled on multiple qubits, which leads to decompositions employing ancilla qubits and controlled single-excitation gates. Ultimately, every quantum computation is eventually compiled down to a physically-accessible set of gates, which may be different from excitation gates. As such, some embodiments are intended not as a compilation method, but rather as a framework for designing quantum algorithms for quantum chemistry. Specifically, for the case of variational quantum algorithms, controlled operations may not be used at all. Quantum circuits capable of preparing approximate eigenstates of molecular Hamiltonians may be constructed, for example, exclusively from two-qubit single excitation gates and four-qubit double-excitation gates. In some implementations, quantum circuits can be designed using Givens rotations directly.

For quantum chemistry applications, custom algorithms are often desired for tackling specific problems and molecules. Using embodiments of the present disclosure, instead of preparing a menu of quantum circuits and algorithms for each instance, scientists/researchers may be presented with a set of universal ingredients (e.g., particle-preserving Givens rotations) that can be employed to craft tailored solutions, via a unifying framework for quantum computational chemistry where every algorithm is a unique recipe built from the same universal ingredients—Givens rotations.

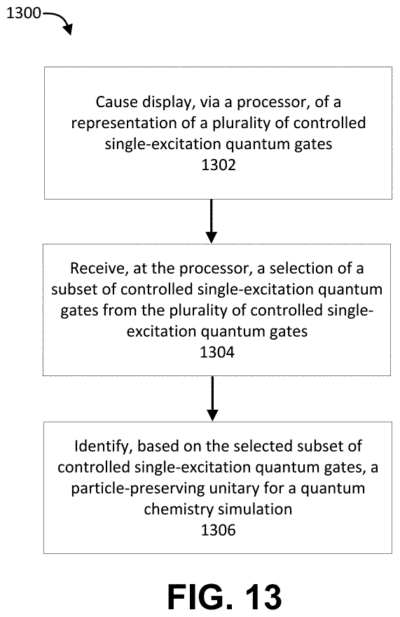

FIGS. 13-16 are flow diagrams showing example quantum chemistry methods, according to some embodiments.

As shown in FIG. 13, a method 1300 includes causing display, at 1302 and via a processor, of representations of each controlled single-excitation quantum gate from a plurality of controlled single-excitation quantum gates. At 1304, a selection of a subset of controlled single-excitation quantum gates from the plurality of controlled single-excitation quantum gates is received at the processor. At 1306, a particle-preserving unitary for a quantum chemistry simulation is identified based on the selected subset of controlled single-excitation quantum gates.

In some implementations, the representation of at least one controlled single-excitation quantum gate from the plurality of controlled single-excitation quantum gates represents a quantum gate that is configured to apply a Givens rotation. In other implementations, each representation of a controlled single-excitation quantum gate from the plurality of controlled single-excitation quantum gates represents a quantum gate that is configured to apply a Givens rotation. In still other implementations, each representation of a controlled single-excitation quantum gate from the plurality of controlled single-excitation quantum gates represents a quantum gate that is configured to implement an arbitrary U(2) transformation on a two-qubit subspace that leaves all other states unchanged, where U is a 2×2 unitary matrix. The quantum gate can be configured to implement the arbitrary U(2) transformation on the two-qubit subspace under control of a control qubit. In still other implementations, each representation of a controlled single-excitation quantum gate from the plurality of controlled single-excitation quantum gates represents a quantum gate that is configured to implement an arbitrary U(2) rotation on a two-qubit subspace that leaves all other states unchanged. In still other implementations, the method 1300 also includes identifying, based on the selected subset of controlled single-excitation quantum gates, a quantum chemistry algorithm that includes the particle-preserving unitary. In still other implementations, the method 1300 also includes receiving, at the processor, a representation of a sequence of controlled single-excitation quantum gates from the subset of controlled single-excitation quantum gates, the identifying the particle-preserving unitary further based on the arrangement of the subset of controlled single-excitation quantum gates from the plurality of controlled single-excitation quantum gates.

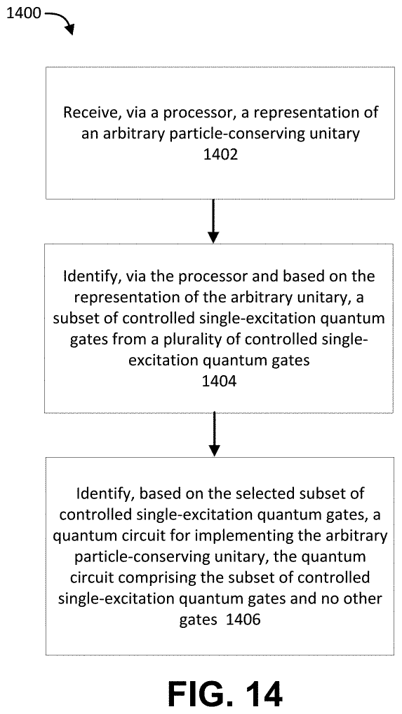

As shown in FIG. 14, a method 1400 includes receiving, at 1402 and via a processor, a representation of an arbitrary particle-conserving unitary. At 1404, a subset of controlled single-excitation quantum gates from a plurality of controlled single-excitation quantum gates is identified via the processor and based on the representation of the arbitrary unitary. At 1406, a quantum circuit for implementing the arbitrary particle-conserving unitary is identified, based on the selected subset of controlled single-excitation quantum gates, the quantum circuit comprising the subset of controlled single-excitation quantum gates and no other gates.

In some implementations, the method 1400 also includes identifying, based on the quantum circuit, a plurality of logic gates to implement the quantum circuit.

In some implementations, at least one controlled single-excitation quantum gate from the subset of controlled single-excitation quantum gates is configured to apply a Givens rotation.

In other implementations, each controlled single-excitation quantum gate from the subset of controlled single-excitation quantum gates is configured to implement an arbitrary U(2) transformation on a two-qubit subspace that leaves all other states unchanged, where U is a 2×2 unitary matrix. In still other implementations, the quantum circuit is configured to approximate an eigenstate of a molecular Hamiltonian.

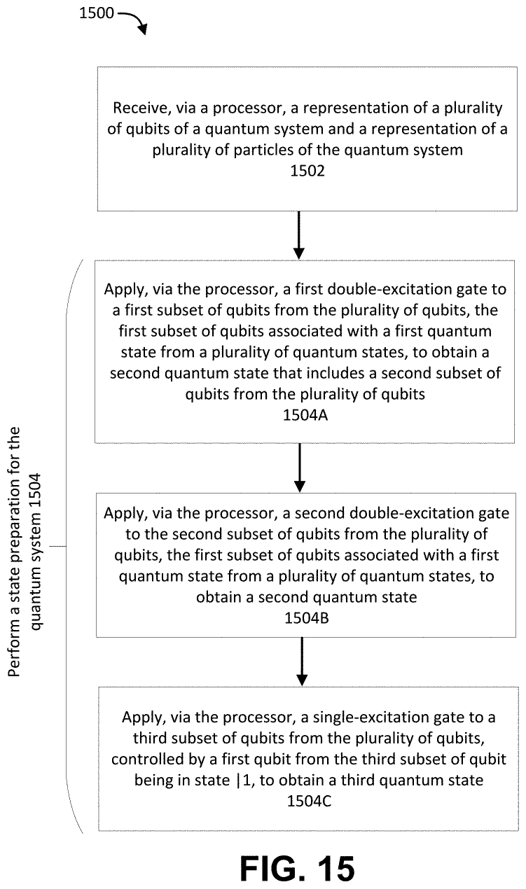

As shown in FIG. 15, a method 1500 includes receiving, at 1502 and via a processor, a representation of a reference state representing an associated plurality of qubits and an associated plurality of particles of a quantum system. At 1504, a state preparation is performed for the quantum system. The state preparation 1504 includes applying, at 1504A and via the processor, a first multi-controlled excitation operation in a subspace between the reference state and a first excited state from a plurality of excited states of the plurality of particles of the quantum system, to obtain a first mapping. The state preparation 1504 also includes applying, at 1504B and via the processor, a second multi-controlled excitation operation in a subspace between the reference state and a second excited state from the plurality of excited states of the plurality of particles of the quantum system, to obtain a second mapping. The state preparation 1504 also includes applying, at 1504C and via the processor, a third multi-controlled excitation operation in a subspace between the reference state and a third excited state from the plurality of excited states of the plurality of particles of the quantum system, to obtain a third mapping. The first mapping, the second mapping, and the third mapping can collectively represent a state preparation of the quantum system.

In some implementations, a number of particles in the plurality of particles is fixed.

In other implementations, at least one of the first multi-controlled excitation operation, the second multi-controlled excitation operation, or the third multi-controlled excitation operation includes a Givens rotation.

In still other implementations, each of the first multi-controlled excitation operation, the second multi-controlled excitation operation, or the third multi-controlled excitation operation includes a Givens rotation.

In still other implementations, the plurality of excited states includes all possible excitations of the reference state.

In other implementations, at least one of the first multi-controlled excitation operation, the second multi-controlled excitation operation, or the third multi-controlled excitation operation includes an arbitrary U(2) transformation on a two-qubit subspace that leaves all other states unchanged, where U is a 2×2 unitary matrix.

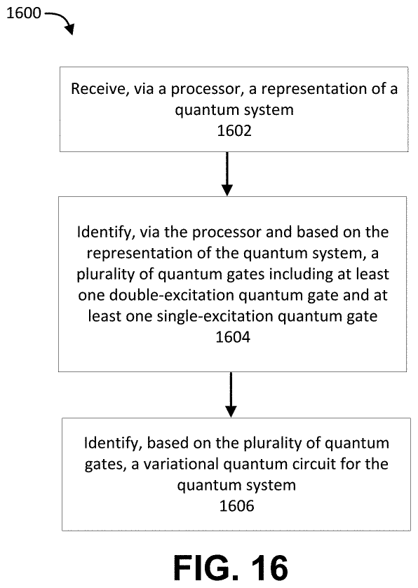

As shown in FIG. 16, a method 1600 includes receiving, at 1602 and via a processor, a representation of a quantum system. At 1604, a plurality of quantum gates including at least one double-excitation quantum gate and at least one single-excitation quantum gate is identified via the processor and based on the representation of the quantum system. At 1606, a variational quantum circuit for the quantum system is identified, based on the plurality of quantum gates.

In some implementations, at least one quantum gate from the plurality of quantum gates is an uncontrolled quantum gate.

In other implementations, at least one quantum gate from the plurality of quantum gates is configured to apply a Givens rotation.

In still other implementations, each quantum gate from the plurality of quantum gates is configured to apply a Givens rotation.

In still other implementations, at least one quantum gate from the plurality of quantum gates is configured to implement an arbitrary U(2) transformation on a two-qubit subspace that leaves all other states unchanged, where U is a 2×2 unitary matrix.

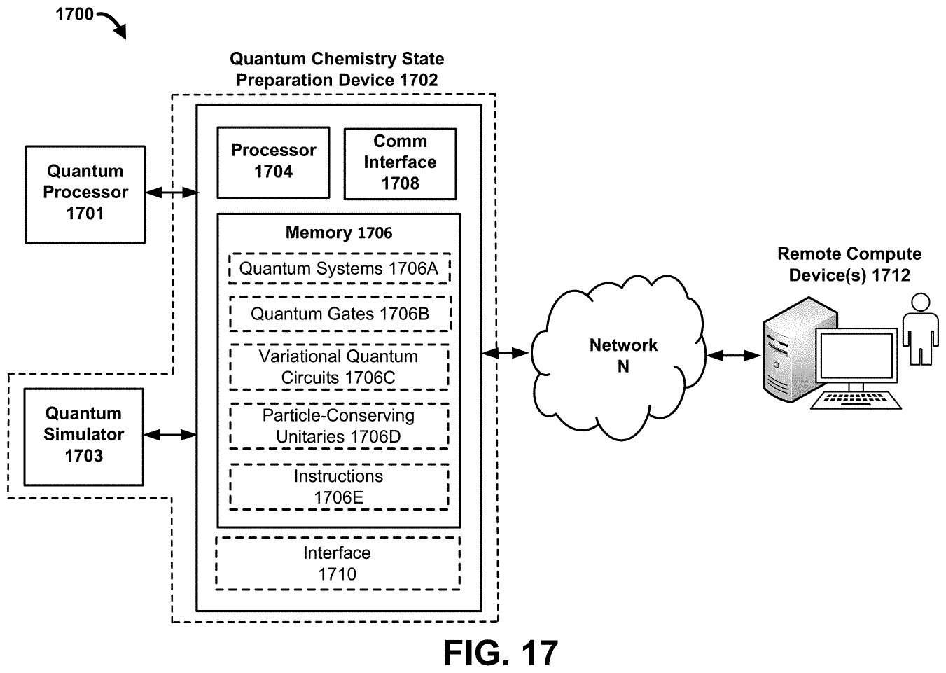

FIG. 17 shows an example system for performing quantum chemistry methods of the present disclosure, in accordance with to some embodiments. As shown in FIG. 17, the system 1700 includes a quantum chemistry state preparation device 1702 including a processor 1704 operably coupled to a memory 1706 and a communications interface 1708. The quantum chemistry state preparation device 1702 is also in communication with (e.g., via a wired and/or wireless communications channel or network) and/or operably coupled to a quantum processor 1701 and a quantum simulator 1703, each of which may include an associated memory and/or set of software instructions. The quantum simulator 1703 may be co-located with (e.g., within a common housing or located at the same geographic location) or remote from (i.e., at a different geographic location from) the quantum chemistry state preparation device 1702. As shown in FIG. 17, the memory 1706 stores one or more of: representations of quantum systems 1706A, representations of quantum gates 1706B (e.g., single-excitation gates and/or double-excitation gates), representations of one or more variational quantum circuits 1706C, and particle-conserving unitaries 1706D. Alternatively or in addition, the memory 1706 can store instructions 1706E, for example to cause the processor to execute the method 1300 of FIG. 13, the method 1400 of FIG. 14, the method 1500 of FIG. 15, and/or the method 1600 of FIG. 16. The quantum chemistry simulator 1702 optionally also includes an interface 1710, which may be implemented in software (e.g., a graphical user interface (“GUI”) for receiving input to the simulation and/or for displaying simulation results) and/or hardware. The quantum chemistry simulator 1702 is configured to communicate via wired and/or wireless communication via the network N, with one or more remote compute devices 1712, for example to receive input to a simulation process.

The quantum processor 1701 and/or the quantum simulator 1703 can be configured to calculate or simulate molecules according to quantum chemistry methods. For example, during operation of the system 1700, in some embodiments, one or more representations of a quantum system (e.g., 1706A), one or more representations of quantum gates (e.g., 1706B), one or more representations of sequences of gates and/or circuits, etc., may be transmitted/sent from the quantum chemistry state preparation device 1702 to the quantum processor 1701, applied or constructed on the quantum processor 1701, and/or executed by the quantum processor 1701 to obtain results that are descriptive of a chemical system of interest. Alternatively or in addition, during operation of the system 1700, one or more representations of a quantum system (e.g., 1706A), one or more representations of quantum gates (e.g., 1706B), one or more representations of sequences of gates and/or circuits, etc., may be transmitted/sent from the quantum chemistry state preparation device 1702 to the quantum simulator 1703, applied or constructed on the quantum simulator 1703, and/or executed by the quantum simulator 1703 to obtain results that are descriptive of a chemical system of interest.

1. A method, comprising:
causing display, via a processor, of a representation of each controlled single-excitation quantum gate from a plurality of controlled single-excitation quantum gates;
receiving, at the processor, a selection of a subset of controlled single-excitation quantum gates from the plurality of controlled single-excitation quantum gates; and
identifying, based on the selected subset of controlled single-excitation quantum gates, a particle-preserving unitary for a quantum chemistry simulation.

2. The method of claim 1, wherein at least one controlled single-excitation quantum gate from the plurality of controlled single-excitation quantum gates is configured to apply a Givens rotation.

3. The method of claim 1, wherein each controlled single-excitation quantum gate from the plurality of controlled single-excitation quantum gates is configured to apply a Givens rotation.

4. The method of claim 1, wherein each controlled single-excitation quantum gate from the plurality of controlled single-excitation quantum gates is configured to implement an arbitrary U(2) transformation on a two-qubit subspace that leaves all other states unchanged, where U is a 2×2 unitary matrix.

5. The method of claim 4, wherein the quantum gate is configured to implement the arbitrary U(2) transformation on the two-qubit subspace under control of a control qubit.

6. The method of claim 1, wherein each controlled single-excitation quantum gate from the plurality of controlled single-excitation quantum gates is configured to implement an arbitrary U(2) rotation on a two-qubit subspace that leaves all other states unchanged.

7. The method of claim 1, further comprising identifying, based on the selected subset of controlled single-excitation quantum gates, a quantum chemistry algorithm that includes the particle-preserving unitary.

8. The method of claim 1, further comprising receiving, at the processor, a representation of a sequence of controlled single-excitation quantum gates from the subset of controlled single-excitation quantum gates, the identifying the particle-preserving unitary further based on the arrangement of the subset of controlled single-excitation quantum gates from the plurality of controlled single-excitation quantum gates.

9. A method, comprising:
receiving, via a processor, a representation of an arbitrary particle-conserving unitary;
identifying, via the processor and based on the representation of the arbitrary unitary, a subset of controlled single-excitation quantum gates from a plurality of controlled single-excitation quantum gates; and
identifying, based on the selected subset of controlled single-excitation quantum gates, a quantum circuit for implementing the arbitrary particle-conserving unitary, the quantum circuit comprising the subset of controlled single-excitation quantum gates and no other gates.

10. The method of claim 9, further comprising identifying, based on the quantum circuit, a plurality of logic gates to implement the quantum circuit.

11. The method of claim 9, wherein at least one controlled single-excitation quantum gate from the subset of controlled single-excitation quantum gates is configured to apply a Givens rotation.

12. The method of claim 9, wherein each controlled single-excitation quantum gate from the subset of controlled single-excitation quantum gates is configured to implement an arbitrary U(2) transformation on a two-qubit subspace that leaves all other states unchanged, where U is a 2×2 unitary matrix.

13. The method of claim 9, wherein the quantum circuit is configured to approximate an eigenstate of a molecular Hamiltonian.

14. A method, comprising:
receiving, via a processor, a representation of a reference state representing an associated plurality of qubits and an associated plurality of particles of a quantum system; and
performing a state preparation for the quantum system, by:
applying, via the processor, a first multi-controlled excitation operation in a subspace between the reference state and a first excited state from a plurality of excited states of the plurality of particles of the quantum system, to obtain a first mapping,
applying, via the processor, a second multi-controlled excitation operation in a subspace between the reference state and a second excited state from the plurality of excited states of the plurality of particles of the quantum system, to obtain a second mapping, and
applying, via the processor, a third multi-controlled excitation operation in a subspace between the reference state and a third excited state from the plurality of excited states of the plurality of particles of the quantum system, to obtain a third mapping.

15. The method of claim 14, wherein a number of particles in the plurality of particles is fixed.

16. The method of claim 14, wherein at least one of the first multi-controlled excitation operation, the second multi-controlled excitation operation, or the third multi-controlled excitation operation includes a Givens rotation.

17. The method of claim 14, wherein the plurality of excited states includes all possible excitations of the reference state.

18. The method of claim 14, wherein at least one of the first multi-controlled excitation, the second multi-controlled excitation, or the third multi-controlled excitation includes an arbitrary U(2) transformation on a two-qubit subspace that leaves all other states unchanged, where U is a 2×2 unitary matrix.

19. A method, comprising:
receiving, via a processor, a representation of a quantum system;
identifying, via the processor and based on the representation of the quantum system, a plurality of quantum gates including at least one double-excitation quantum gate and at least one single-excitation quantum gate; and
identifying, based on the plurality of quantum gates, a variational quantum circuit for the quantum system.

20. The method of claim 19, wherein at least one quantum gate from the plurality of quantum gates is an uncontrolled quantum gate.

21. The method of claim 19, wherein at least one quantum gate from the plurality of quantum gates is configured to apply a Givens rotation.

22. The method of claim 19, wherein each quantum gate from the plurality of quantum gates is configured to apply a Givens rotation.

23. The method of claim 19, wherein at least one quantum gate from the plurality of quantum gates is configured to implement an arbitrary U(2) transformation on a two-qubit subspace that leaves all other states unchanged, where U is a 2×2 unitary matrix.
